(19) United States
(12) Patent Application Publication
(10) Pub. No.: US 2020/0156084 A1
(43) Pub. Date: May 21, 2020

(54) GAS PURIFYING DEVICE

(71) Applicant: Microjet Technology Co., Ltd., Hsinchu (TW)

(72) Inventors: Hao-Jan Mou, Hsinchu (TW); Ching-Sung Lin, Hsinchu (TW); Chin-Chuan Wu, Hsinchu (TW); Chih-Kai Chen, Hsinchu (TW); Chi-Feng Huang, Hsinchu (TW); Yung-Lung Han, Hsinchu (TW); Hsuan-Kai Chen, Hsinchu (TW)

(73) Assignee: Microjet Technology Co., Ltd., Hsinchu (TW)

(21) Appl. No.: 16/683,679

(22) Filed: Nov. 14, 2019

(30) Foreign Application Priority Data
Nov. 16, 2018 (TW) 107140928

(51) Int. Cl. B03C 3/32; B01D 46/24; G01N 33/00

(52) U.S. Cl. CPC B03C 3/32; B01D 46/2403; F24F 2221/12; B01D 2273/30; G01N 33/0027

(57) ABSTRACT

A gas purifying device is disclosed and comprises a gas purifier and a gas detector. The gas purifier comprises a purifier main body, a filter, an air guiding device and a drive control module. The purifier main body has an embedding slot. The gas detector is assembled in the embedding slot for or detached from the embedding slot for independently use. The gas detector comprises a gas detecting module, a particulate measuring module and a detector drive control module. The gas detecting module comprises a gas sensor and a gas actuator. The particulate measuring module comprises a particulate detector and a particulate actuator. The detector drive control module controls the actuation of the gas detecting module and the particulate measuring module and converts the monitored information from the gas detecting module and the particulate measuring module into a monitored data information, and outputs the monitored data information.

### FIELD OF THE INVENTION

The present disclosure relates to a gas purifying device, and more particularly to a thin and portable gas purifying device capable of monitoring gas.

### BACKGROUND OF THE INVENTION

Nowadays, people pay much attention to the air quality in the environment. For example, it is important to monitor carbon monoxide, carbon dioxide, volatile organic compounds (VOC), Particulate Matter 2.5 (PM2.5), nitric oxide, sulfur monoxide, and so on. The exposure of these substances in the environment will cause human health problems or even harm the life. Therefore, it is important for every country to monitor the air quality in the environment, which is a topic currently being valued.

Generally, it is feasible to use a gas sensor to monitor the air quality in the environment. If the gas sensor is capable of immediately providing people with the monitored information relating to the environment for caution, it may help people escape or prevent from the injuries and influence on human health caused by the exposure of the substances described above in the environment. In other words, the gas sensor is suitably used for monitoring the ambient air in the environment. A gas purifying device is a solution for reducing air pollution and protecting people away from harmful gas. Therefore, how to provide a gas purifying device in combination with a gas detection device for monitoring gas immediately everywhere and anytime and achieving benefits of purifying gas to improve air quality, are main subjects of research and development in present application.

### SUMMARY OF THE INVENTION

An object of the present disclosure provides a gas purifying device in combination with a gas detector. The gas purifying device utilizes a gas detection module and a particle detection module to monitor air quality around a user so as to achieve the purpose of being able to carry the gas purifying device around and monitor anywhere and anytime, thereby providing the benefits of monitoring rapidly and accurately. Consequently, air quality information is obtained in real time and is provided as a notification to the user in the environment, so that the user can prevent or escape from the injuries and influence on human health caused by the exposure of the harmful substances in the environment. In addition, the gas purifying device utilizes a gas purifying device to achieve the benefits of purifying gas so as to improve air quality.

In accordance with an aspect of the present disclosure, there is provided a gas purifying device including a gas purifier and a gas detector. The gas purifier comprises a purifier main body, a filter, an air guiding device and a drive control module and configured to purify gas. The gas detector comprises a gas detecting module, a particulate measuring module and a detector drive control module. The gas detecting module comprises a gas sensor and a gas actuator, wherein the gas actuator controls the gas to be guided to the interior of the gas detecting module and pass through the gas sensor for gas detecting. The particulate measuring module comprises a particulate detector and a particulate actuator, wherein the particulate actuator controls the gas to be guided to the interior of the particulate measuring module, and the particulate detector measures the sizes and the concentrations of the suspended particles contained in the gas. The detector drive control module controls the actuation of the gas detecting module and the particulate measuring module, converts monitored information from the gas detecting module and the particulate measuring module into a monitored data information, and outputs the monitored data information.

The above contents of the present disclosure will become more readily apparent to those ordinarily skilled in the art after reviewing the following detailed description and accompanying drawings, in which:

### BRIEF DESCRIPTION OF THE DRAWINGS

FIG. 1A is a schematic perspective view illustrating a gas purifying device according to an embodiment of the present disclosure;

FIG. 1B is a schematic exploded view illustrating the gas purifying device of the present disclosure;

FIG. 2A is a schematic cross-sectional view illustrating gas flow direction of the gas purifying device of the present disclosure and taken at a viewpoint;

FIG. 2B is a schematic cross-sectional view illustrating gas flow direction of the gas purifying device of the present disclosure and taken at another viewpoint;

FIG. 3A is a schematic perspective view illustrating a gas detector of the gas purifying device according to an embodiment of the present disclosure;

FIG. 3B is a front view illustrating the gas detector of the gas purifying device of the present disclosure;

FIG. 3C is a right side view illustrating the gas detector of the gas purifying device of the present disclosure;

FIG. 3D is a left side view illustrating the gas detector of the gas purifying device of the present disclosure;

FIG. 3E is a schematic cross-sectional view illustrating the gas detector of the gas purifying device of the present disclosure;

FIG. 4A is a front view illustrating the gas detecting module of the gas purifying device of the present disclosure;

FIG. 4B is a rear view illustrating the gas detecting module of the gas purifying device of the present disclosure;

FIG. 4C is a schematic exploded view illustrating the gas detecting module of the gas purifying device of the present disclosure;

FIG. 4D is a partially enlarged schematic cross-sectional view illustrating a gas flow direction of the gas detecting module of the gas purifying device of the present disclosure;

FIG. 4E is a schematic perspective view illustrating the gas flow direction of the gas detecting module of the gas purifying device of the present disclosure;

FIG. 5 is a schematic perspective view illustrating a particulate measuring module and a detector drive control module of the gas purifying device of the present disclosure;

FIG. 6 is a schematic cross-sectional view illustrating the particulate measuring module of the gas purifying device of the present disclosure;

FIG. 7A is a schematic exploded view illustrating a miniature pump of the gas detecting module of the present disclosure;

FIG. 7B is a schematic exploded view illustrating the miniature pump of the gas detecting module of the present disclosure and taken at another viewpoint;

FIG. 8A is a schematic cross-sectional view illustrating the miniature pump of the gas detecting module of the present disclosure;

FIG. 8B is a schematic cross-sectional view illustrating the miniature pump of the gas detecting module according to another embodiment of the present disclosure;

FIGS. 8C, 8D and 8E schematically illustrate the actions of the miniature pump of the gas detecting module of the present disclosure;

FIG. 9 is a schematic exploded view illustrating the micro box pump of the gas purifying device of the present disclosure;

FIGS. 10A, 10B and 10C schematically illustrate the actions of the micro box pump of the present disclosure; and

FIG. 11 schematically illustrates the signal communication and transmission of the gas purifying device of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed.

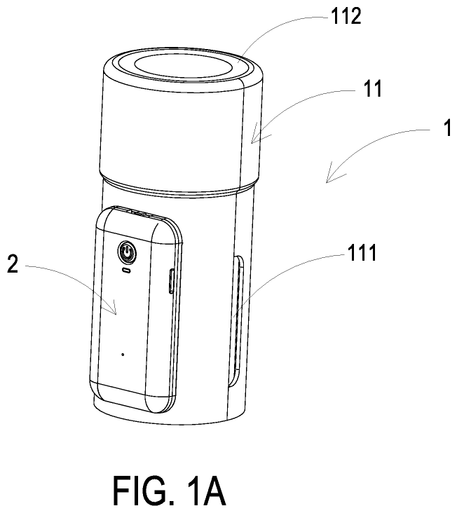

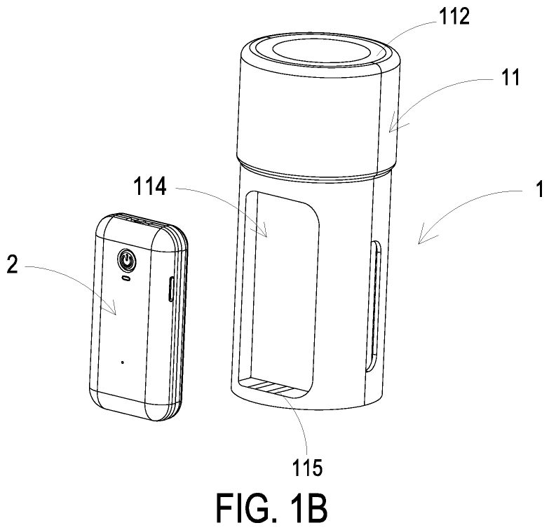

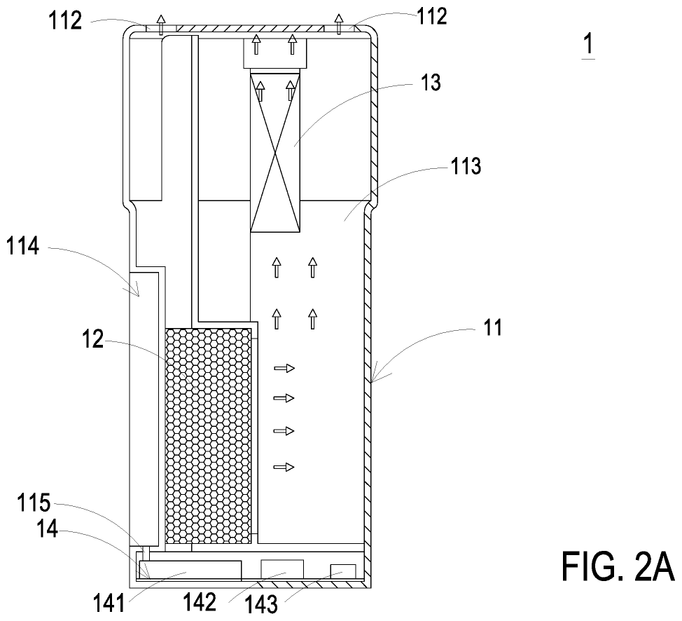

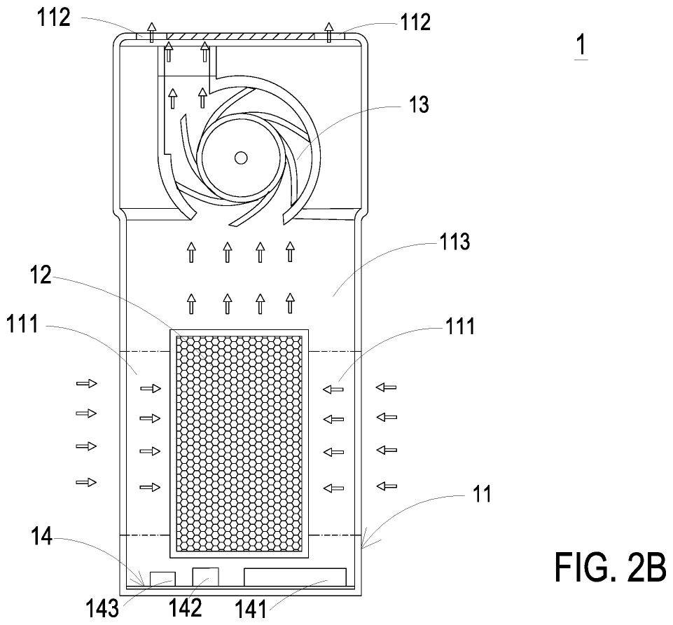

Please refer to FIGS. 1A, 1B, 2A and 2B. The present disclosure provides a gas purifying device including a gas purifier 1 and a gas detector 2. The gas purifier 1 includes a purifier main body 11, a filter 12, an air guiding device 13 and a drive control module 14. The purifier main body 11 has at least one inlet 111 and an outlet 112 disposed on the exterior thereof and has a guiding channel 113 disposed in the interior thereof and in fluid communication between the inlet 111 and the outlet 112. The filter 12 is disposed between the inlet 111 and the guiding channel 113 and allows the gas to be purified to pass therethrough and flow into the guiding channel 113. The air guiding device 13 is disposed between the outlet 112 and the guiding channel 113 and transports and guides the gas inside the guiding channel 113 to be discharged via the outlet 112. While the air guiding device 13 is driven, the air guiding device 13 pumps the gas inside the guiding channel 113 that makes the external gas inhaled via the inlet 111, passes through the filter 12 and is purified thereby. After that, the purified gas is transported to the guiding channel 113 and then discharged via the outlet 112, so that the user can breathe clean gas. In some embodiments, the gas detector 2 is directly disposed in the interior of the purifier main body 11. In this embodiment, the purifier main body 11 has an embedding slot 114 concavely formed on the exterior thereof. The gas detector 2 is assembled in the embedding slot 114 to be positioned, or the gas detector 2 is detached from the embedding slot 114 and configured for use independently. The drive control module 14 is disposed in the interior of the purifier main body 11. A connection port 115 is disposed in the embedding slot 114 and electrically connected to the drive control module 14. While the gas detector 2 is assembled and positioned in embedding slot 114, the gas detector 2 and the drive control module 14 are electrically connected to each other through the connection port 115 for allowing electrical power and signal to be transmitted therebetween. In the embodiment, the filter 12 is an electrostatic filter, an activated carbon filter or a High-Efficiency Particulate Air (HEPA) filter.

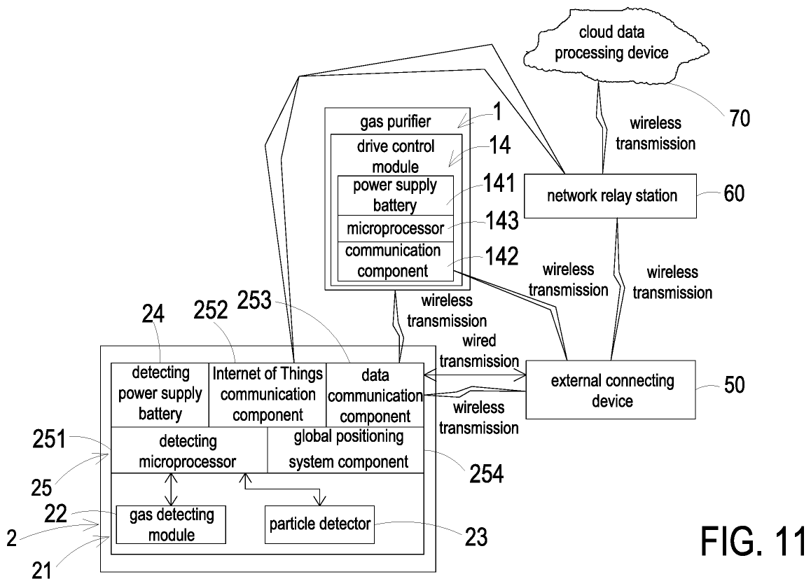

Please refer to FIGS. 2A, 2B and 11. The drive control module 14 includes a power supply battery 141, a communication component 142 and a microprocessor 143. The power supply battery 141 is electrically connected to a power source for storing electrical energy therein and outputs electrical energy to the microprocessor 143 and the air guiding device 13. The power supply battery 141 is electrically connected to the power source by means of a wired transmission or a wireless transmission so as to charge and store electrical energy. The communication component 142 receives the monitored information from the gas detector 2 or receives a transmission signal from an external connecting device 50 via a wireless communication technology. Then, the monitored information or the transmission signal is transmitted to the microprocessor 143 and converted into a control signal by the microprocessor 143 so that the activation of the air guiding device 13 is controlled to activate the gas purifier 1 to purify gas.

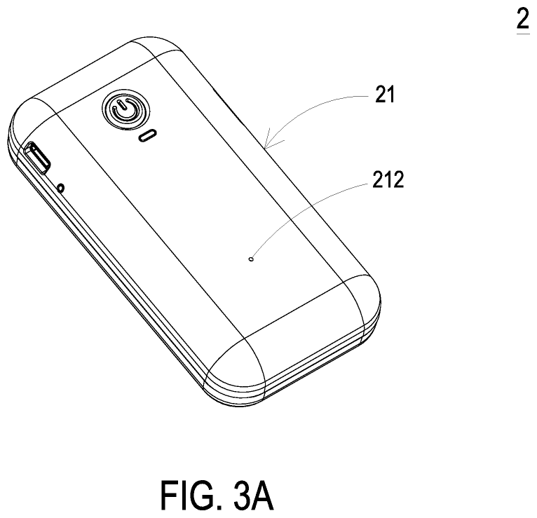

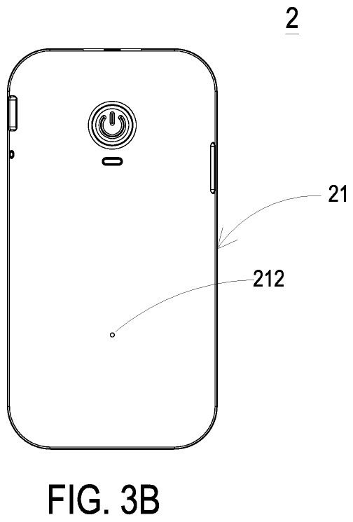

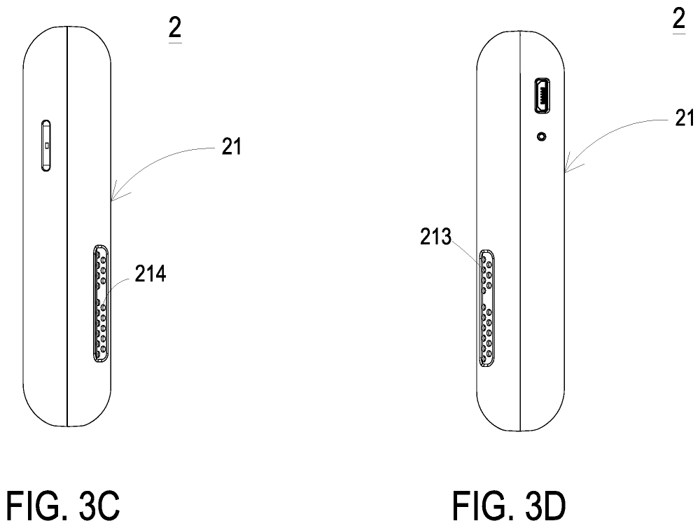

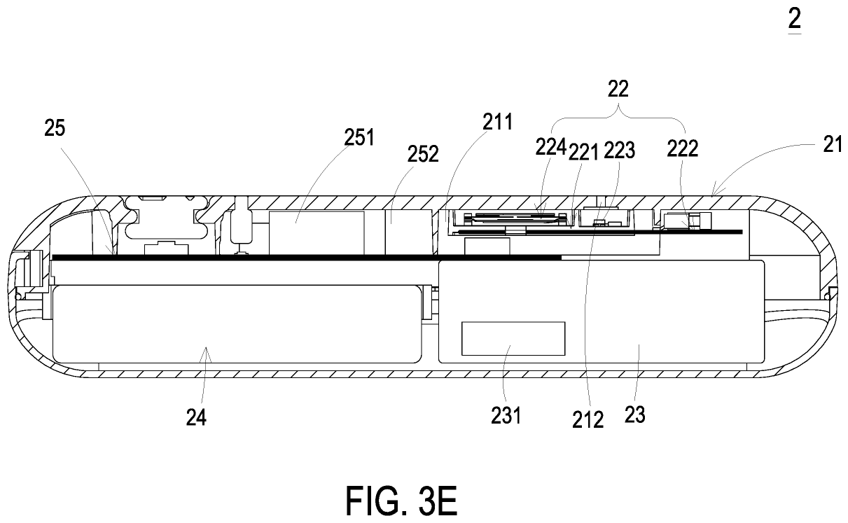

Please refer to FIGS. 3A to 3E, 4A to 4E, 5 and 6. The gas detector 2 includes a detector main body 21, a gas detecting module 22, a particulate measuring module 23, a detecting power supply battery 24 and a detector drive control module 25. The detector main body 21 includes a chamber 211 disposed in the interior thereof and has a first inlet 212, a second inlet 213 and a detecting outlet 214 disposed on the exterior thereof and in fluid communication with the chamber 211.

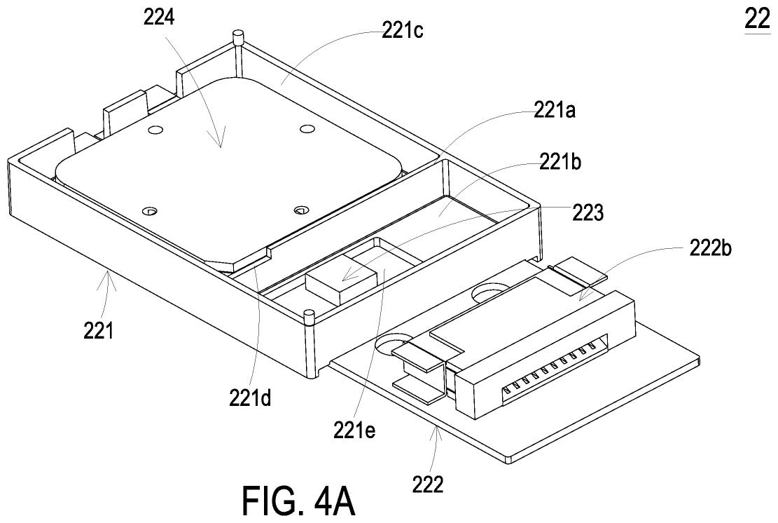

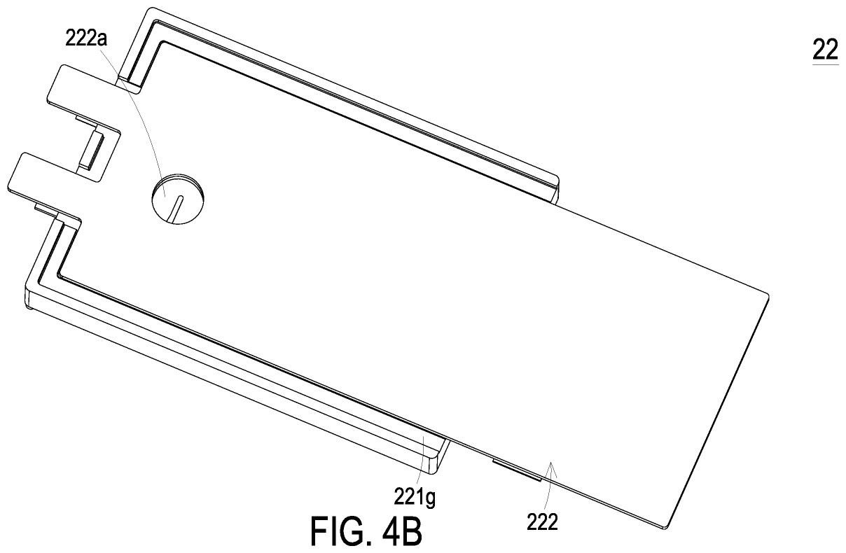

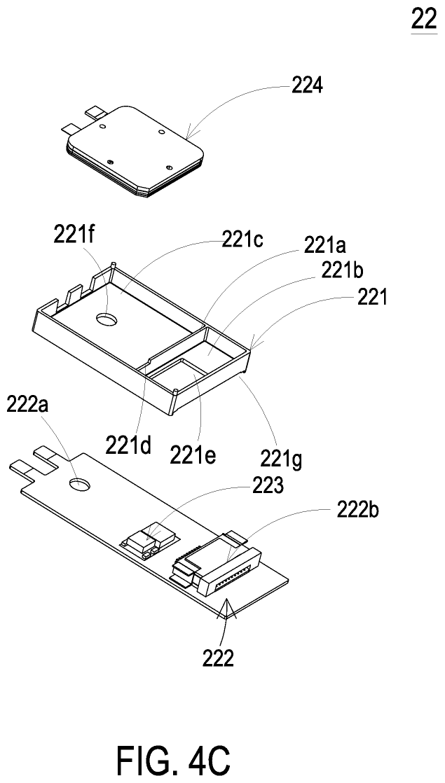

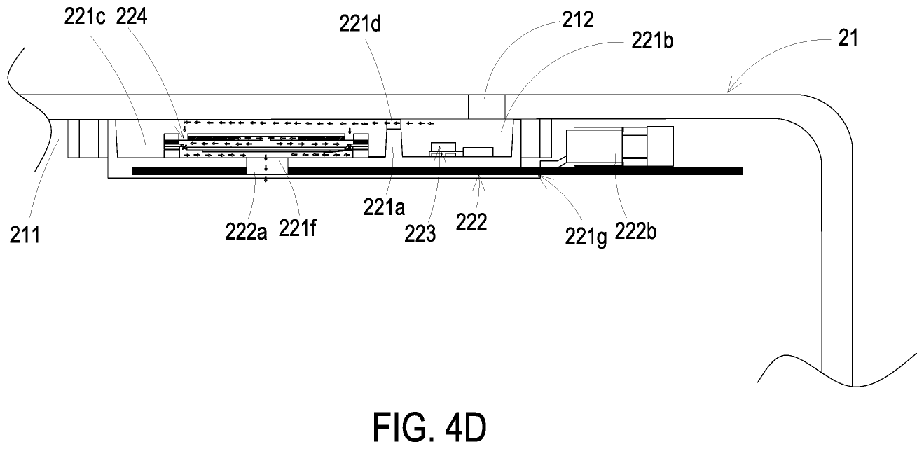

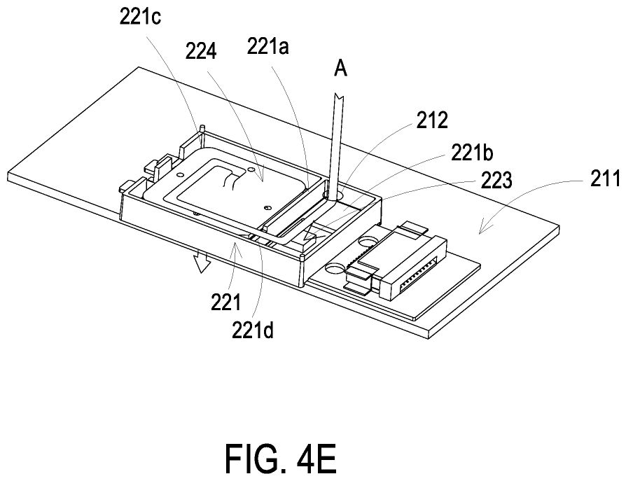

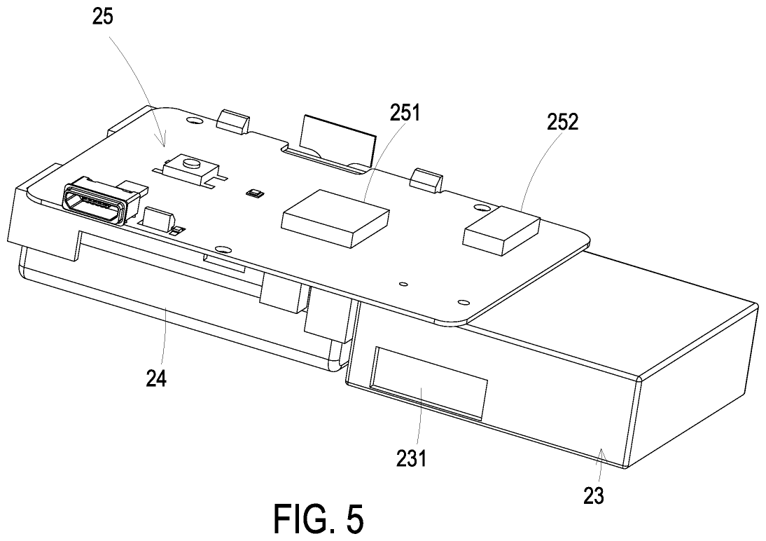

Please refer to FIG. 3E and FIGS. 4A to 4E. The gas detecting module 22 includes a compartment body 221, a carrier 222, a gas sensor 223 and a gas actuator 224. The compartment body 221 is disposed under the first inlet 212 of the detector main body 21, and has a partition 221a which divides the internal of the compartment body 221 into a first gas compartment 221b and a second gas compartment 221c. The partition 221a has a notch 221d for allowing the first gas compartment 221b and the second gas compartment 221c to be in fluid communication with each other. The first gas compartment 221b has an opening 221e, the second gas compartment 221c has a discharging opening 221f, and an accommodation groove 221g is disposed on the bottom of the compartment body 221. The carrier 222 is positioned in the accommodation groove 221g so as to enclose the bottom of the compartment body 221. The carrier 222 has a ventilation hole 222a, and the gas sensor 223 is packaged on and electrically connected to the carrier 222. When the carrier 222 is disposed under the compartment body 221, the ventilation hole 222a is corresponding in position to the discharging opening 221f of the second gas compartment 221c, and the gas sensor 223 is accommodated in the first gas compartment 221b through the opening 221e so as to monitor the gas in the first gas compartment 221b. The gas actuator 224 is disposed in the second gas compartment 221c and is insulated from the gas sensor 223 disposed in the first gas compartment 221b. The heat generated by the gas actuator 224 while actuated can be blocked by the partition 221a so as to avoid affecting the detection result of the gas sensor 223. The gas actuator 224 encloses the bottom of the second gas compartment 221c, and is controlled to generate a gas flow. The gas flow is discharged from the compartment body 221 via the discharging opening 221f of the second gas compartment 221c, and finally discharged from the gas detecting module 22 via the ventilation hole 222a of the carrier 222. The carrier 222 may be a print circuit board having a connector 222b, and a circuit board (not shown in the figures) is connected to the connector 222b, so that the detector drive control module 25 (shown in FIG. 5) is in electrical and signal connection to the carrier 222.

Please refer to FIGS. 4A, 4D and 4E. For ease of reference of the gas flow direction inside the gas detecting module 22, the detector main body 21 is shown in perspective view. When the gas detecting module 22 is disposed in the chamber 211 of the detector main body 21, the first inlet 212 of the detector main body 21 is corresponding to the first gas compartment 221b of the compartment body 221. In the embodiment, the first inlet 212 of the detector main body 21 is not directly corresponding in position to the gas sensor 223 disposed in the first gas compartment 221b. In other words, the first inlet 212 is not disposed above the gas sensor 223, which means the first inlet 212 and the gas sensor 223 are misaligned. By controlling the actuation of the gas actuator 224, a negative pressure is formed inside the second gas compartment 221c, so that gas is inhaled from the external of the detector main body 21. After that, gas is guided into the first gas compartment 221b and passes through the surface of the gas sensor 223 for gas detection, so as to detect the air quality of the external of the detector main body 21. When the gas actuator 224 operates continuously, the detected gas is guided to the second gas compartment 221c through the notch 221d of the partition 221a, and is finally discharged from the compartment body 221 via the discharging opening 221f and the ventilation hole 222a of the carrier 222, so as to constitute a one-way gas transportation monitoring (shown as an airflow path A in FIG. 4E).

The gas sensor 223 may be at least one selected from the group consisting of an oxygen sensor, a carbon monoxide sensor, a carbon dioxide sensor, a temperature sensor, an ozone sensor, a volatile organic compound (VOC) sensor and combinations thereof. In some embodiments, the gas sensor 223 may be at least one selected from the group consisting of a bacterial sensor, a virus sensor, a microorganism sensor and combinations thereof.

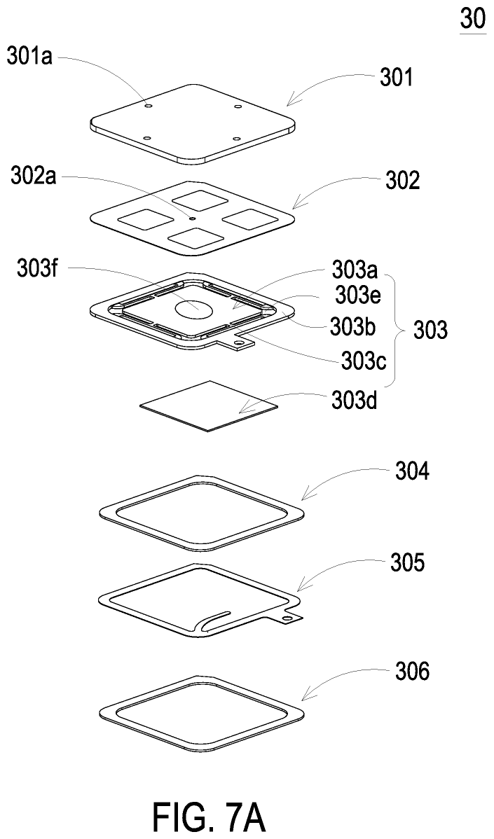

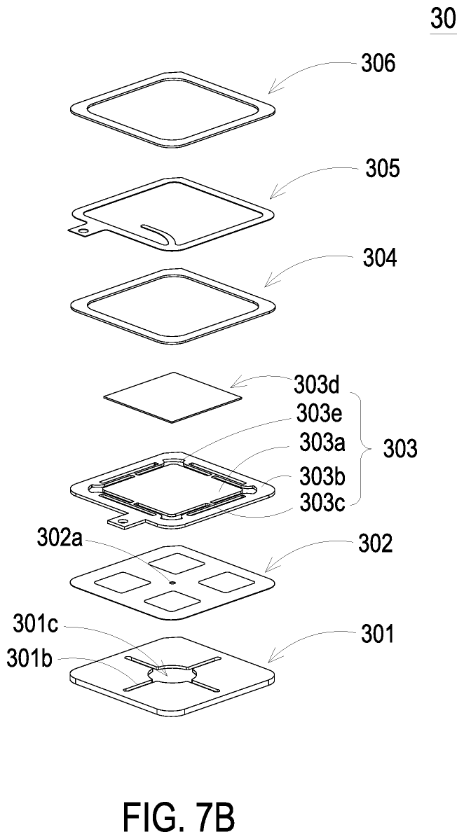

Please refer to FIGS. 7A to 7B. The gas actuator 224 is a miniature pump 30. The miniature pump 30 includes a gas inlet plate 301, a resonance plate 302, a piezoelectric actuator 303, a first insulation plate 304, a conducting plate 305 and a second insulation plate 306, which are stacked on each other sequentially. The gas inlet plate 301 has at least one inlet aperture 301a, at least one convergence channel 301b and a convergence chamber 301c. The inlet aperture 301a allows gas to be introduced therethrough, the inlet aperture 301a runs through the gas inlet plate 301 and is corresponding in position to the convergence channel 301b, and the convergence channel 301b converges to the convergence chamber 301c, so that gas inhaled via the inlet aperture 301a is converged to the convergence chamber 301c. In the embodiment, the number of the inlet aperture 301a is equal to the number of the convergence channel 301b. In this embodiment, the number of the inlet aperture 301a and the convergence channel 301b is exemplified by four for each, but not limited thereto. The four inlet apertures 301a are in fluid communication with the four convergence channels 301b, respectively, and the four convergence channels 301b converge to the convergence chamber 301c.

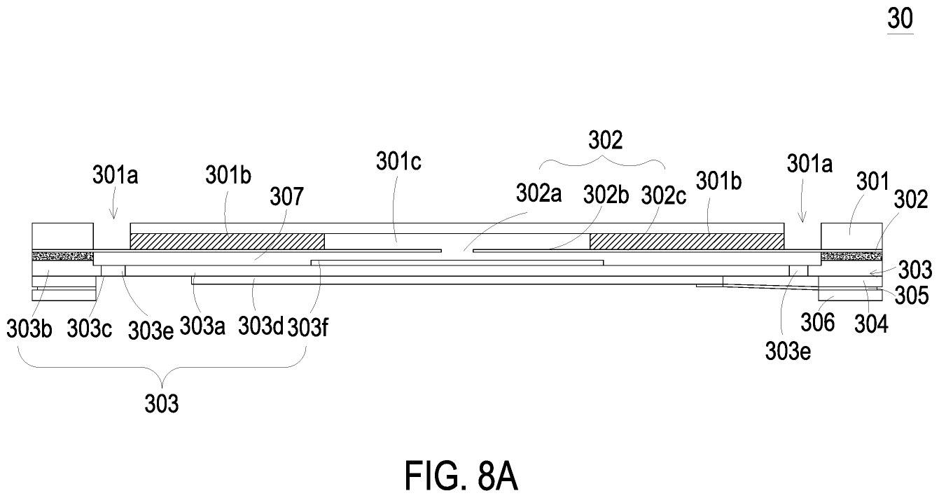

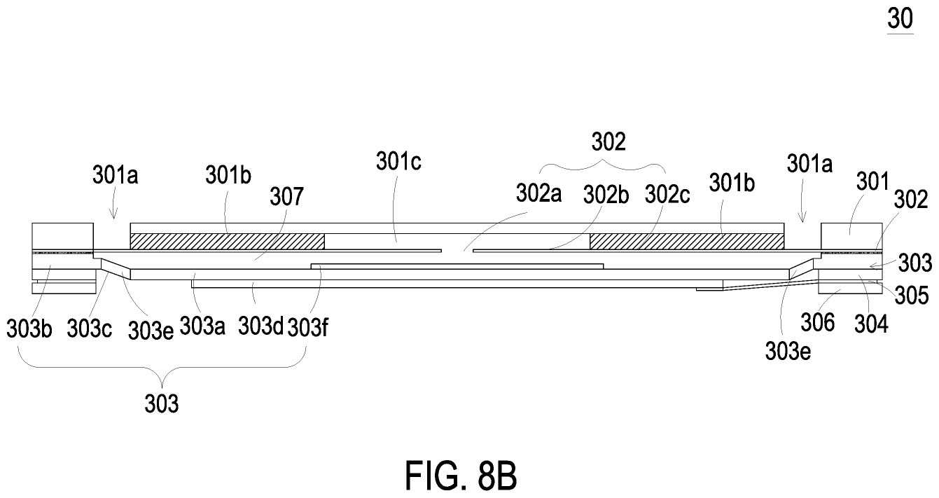

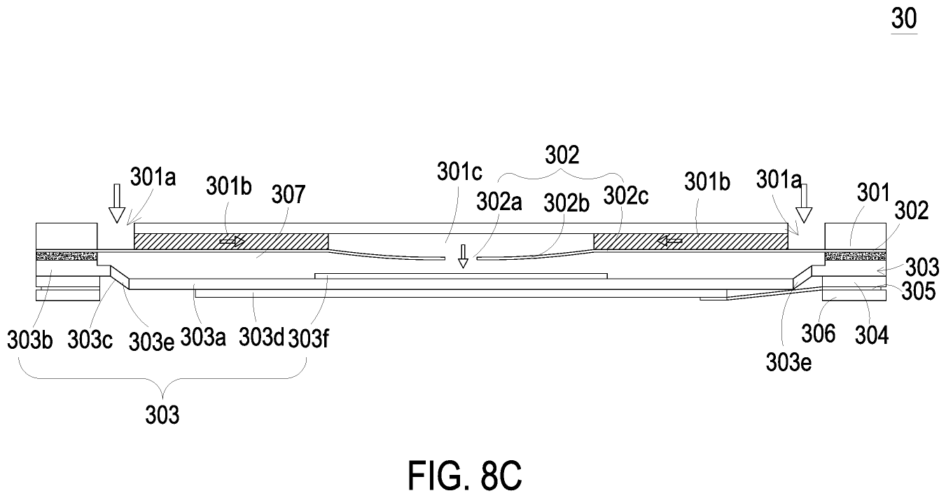

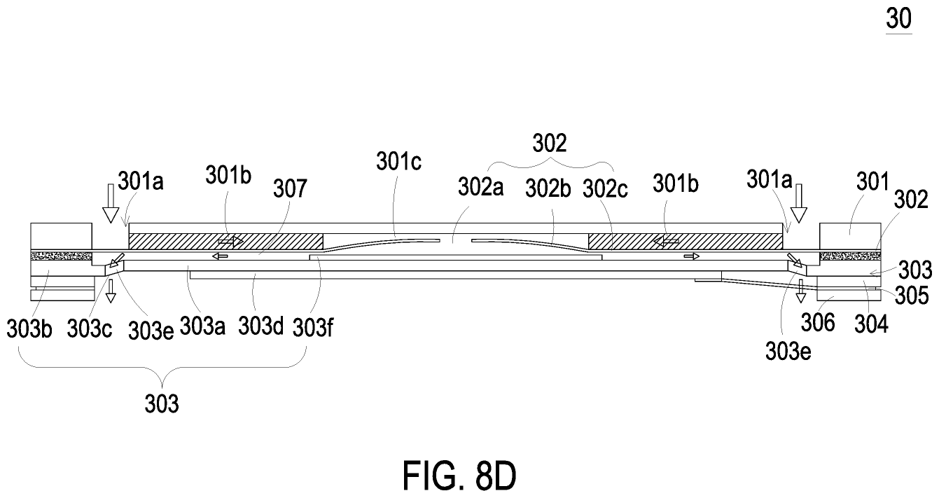

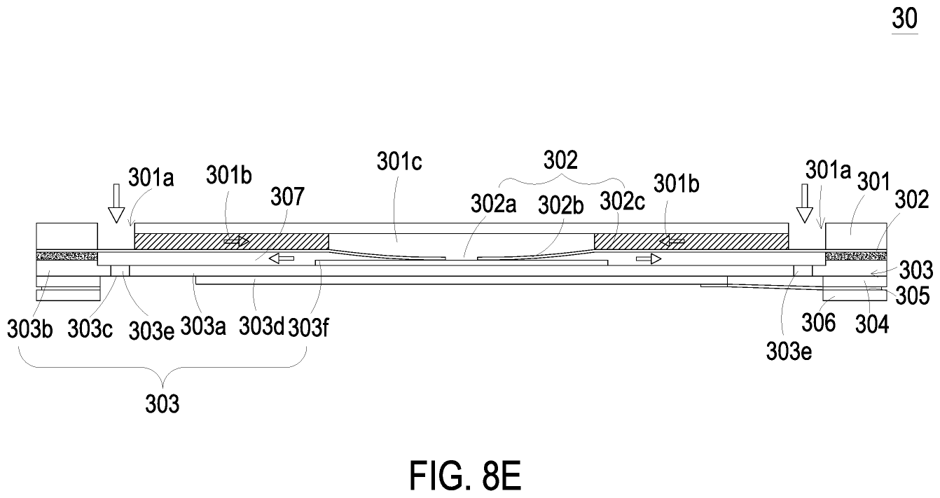

Please refer to FIGS. 7A, 7B and 8. The resonance plate 302 is assembled on the gas inlet plate 301 by attaching means. The resonance plate 302 has a central aperture 302a, a movable part 302b and a fixed part 302c. The central aperture 302a is located in the center of the resonance plate 302 and is corresponding in position to the convergence chamber 301c of the gas inlet plate 301. The region of the resonance plate 302 around the central aperture 302a and corresponding in position to the convergence chamber 301c is the movable part 302b. The region of the periphery of the resonance plate 302 securely attached on the gas inlet plate 301 is the fixed part 302c.

Please refer to FIGS. 7A, 7B and 8A continuously. The piezoelectric actuator 303 includes a suspension plate 303a, an outer frame 303b, at least one bracket 303c, a piezoelectric element 303d, at least one vacant space 303e and a bulge 303E The suspension plate 303a is a square suspension plate. By comparing with a circle suspension plate, the square suspension plate 303a obviously has the power saving advantage. Considering the capacitive load during the period of operation under the resonant frequency, as the resonant frequency increases, and so does the power consumption. Further, since the resonant frequency of the square suspension plate 303a is lower than that of the circle suspension plate, the relative power consumption of the square suspension plate 303a is obviously reduced. In other words, the square suspension plate 303a of the present disclosure achieves the power saving advantage. The outer frame 303b is disposed around the periphery of the suspension plate 303a. The at least one bracket 303c is connected between the suspension plate 303a and the outer frame 303b for elastically supporting the suspension plate 303a. In the embodiment, a length of a side of the piezoelectric element 303d is smaller than or equal to a length of a side of the suspension plate 303a. The piezoelectric element 303d is attached on a surface of the suspension plate 303a for driving the suspension plate 303a to undergo the bending vibration in response to an applied voltage. The at least one vacant space 303e is formed among the suspension plate 303a, the outer frame 303b and the at least one bracket 303c for allowing the gas to flow therethrough. The bulge 303f is disposed on a surface of the suspension plate 303a opposite to the surface attaching the piezoelectric element 303d. In this embodiment, the bulge 303f is formed on the suspension plate 303a by an etching process and is a convex structure integrally formed on the surface of the suspension plate 303a opposite to the surface attaching the piezoelectric element 303d.

Please also refer to FIGS. 7A, 7B and 8. The gas inlet plate 301, the resonance plate 302, the piezoelectric actuator 303, the first insulation plate 304, the conducting plate 305 and the second insulation plate 306 are stacked sequentially. A chamber space 307 is formed between the suspension plate 303a and the resonance plate 302. The chamber space 307 between the suspension plate 303a and the resonance plate 302, may be filled with a filler, for example but not limited to a conductive adhesive, so that a specific depth between the suspension plate 303a and the resonance plate 302 can be maintained. The chamber space 307 ensures the proper distance between the suspension plate 303a and the resonance plate 302, so that the gas can be transported more rapidly and the contact interference and the generated noise are largely reduced. In some embodiments, alternatively, the height of the outer frame 303b of the piezoelectric actuator 303 is increased. Accordingly, the thickness of the conductive adhesive filled within the gap between the resonance plate 302 and the outer frame 303b of the piezoelectric actuator 303 is reduced. Therefore, in case that the suspension plate 303a and the resonance plate 302 are maintained at a proper distance, the thickness of the conductive adhesive filled within the overall assembly of the miniature pump 30 won't be affected by a hot pressing temperature and a cooling temperature. It benefits to avoid that the conductive adhesive affects the actual size of the chamber space 307 due to the factors of thermal expansion and contraction after the assembly is completed. The present disclosure is not limited thereto. In addition, since the transportation efficiency of the miniature pump 30 is affected by the chamber space 307, it is important to maintain the chamber space 307 in a specific depth for the miniature pump 30 to provide stable transportation efficiency.

Please refer to FIG. 8B. In another exemplary structure of the piezoelectric actuator 303, the suspension plate 303a may be formed by a stamping method. The stamping method makes the suspension plate 303a extended outwardly at a distance. The distance extended outwardly may be adjusted by the bracket 303c formed between the suspension plate 303a and the outer frame 303b, so that a surface of the bulge 303f on the suspension plate 303a is not coplanar with a surface of the outer frame 303b. A small amount of material (e.g., conductive adhesive) is applied to the assembling surface of the outer frame 303b for attaching the piezoelectric actuator 303 on the fixed part 302c of the resonance plate 302 by means of a hot pressing process. Further, the piezoelectric actuator 303 is assembled with the resonance plate 302. In this way, the entire structure may be improved by adopting the stamping method to form the suspension plate 303a of the piezoelectric actuator 303, thereby modifying the chamber space 307. A desired size of the chamber space 307 may be satisfied by simply adjusting the distance from the resonance plate 302 to the suspension plate 303a of the piezoelectric actuator 303 through the stamping method. It simplifies the structural design for adjusting the chamber space 307. At the same time, it achieves the advantages of simplifying the process and saving the process time. In the embodiment, the first insulation plate 304, the conducting plate 305 and the second insulation plate 306 are all frame-shaped thin sheet and are stacked sequentially on the piezoelectric actuator 303 to obtain the entire structure of the miniature pump 30.

For describing the actions of the miniature pump 30, please refer to FIGS. 8C to 8E. Firstly, as shown in FIG. 8C, when the piezoelectric element 303d of the piezoelectric actuator 303 is deformed in response to an applied voltage, the suspension plate 303a is displaced in a direction away from the gas inlet plate 301. In that, the volume of the chamber space 307 is increased, a negative pressure is formed in the chamber space 307, and the gas in the convergence chamber 301c is inhaled into the chamber space 307. At the same time, the resonance plate 302 is in resonance and thus displaced synchronously in the direction away from the gas inlet plate 301. Thereby, the volume of the convergence chamber 301c is increased. Since the gas in the convergence chamber 301c flows into the chamber space 307, the convergence chamber 301c is also in a negative pressure state, and the gas is sucked into the convergence chamber 301c by flowing through the inlet aperture 301a and the convergence channel 301b. Then, as shown in FIG. 8D, the piezoelectric element 303d drives the suspension plate 303a to be displaced toward the gas inlet plate 301 to compress the chamber space 307. Similarly, the resonance plate 302 is actuated by the suspension plate 303a (i.e., in resonance with the suspension plate 303a) and is displaced toward the gas inlet plate 301. Thus, the gas in the chamber space 307 is compressed synchronously and forced to be further transported through the vacant space 303e to achieve the effect of gas transportation. Finally, as shown in FIG. 8E, when the suspension plate 303a is vibrated back to the initial state, which is not driven by the piezoelectric element 303d, the resonance plate 302 is also driven to displace in the direction away from the gas inlet plate 301 at the same time. In that, the resonance plate 302 pushes the gas in the chamber space 307 toward the vacant space 303e, and the volume of the convergence chamber 301c is increased. Thus, the gas continuously flows through the inlet aperture 301a and the convergence channel 301b and is converged in the confluence chamber 301c. By repeating the actions of the miniature pump 30 shown in the above-mentioned FIGS. 8C to 8E continuously, the miniature pump 30 can continuously transport the gas at a high speed to accomplish the gas transportation and output operations of the miniature pump 30.

Please refer to FIG. 8A. In the embodiment, the gas inlet plate 301, the resonance plate 302, the piezoelectric actuator 303, the first insulation plate 304, the conducting plate 305 and the second insulation plate 306 of the miniature pump 30 are all produced by a micro-electromechanical surface micromachining technology. Thereby, the volume of the miniature pump 30 is reduced, and a micro-electromechanical system of the miniature pump 30 is constructed.

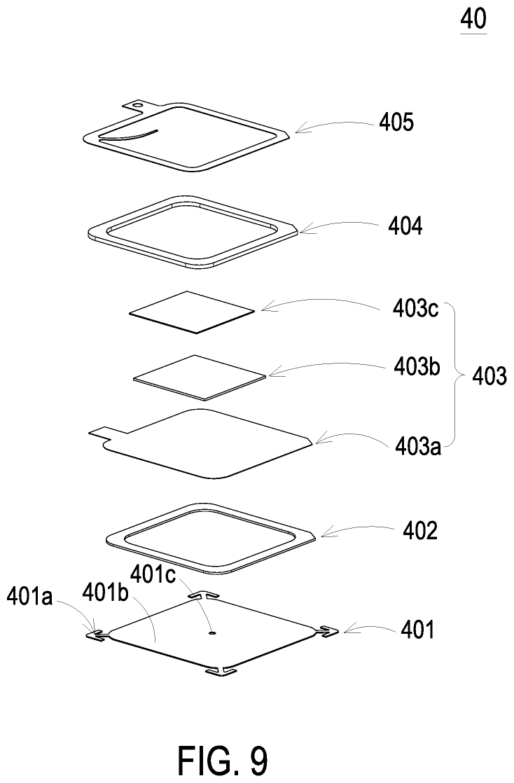

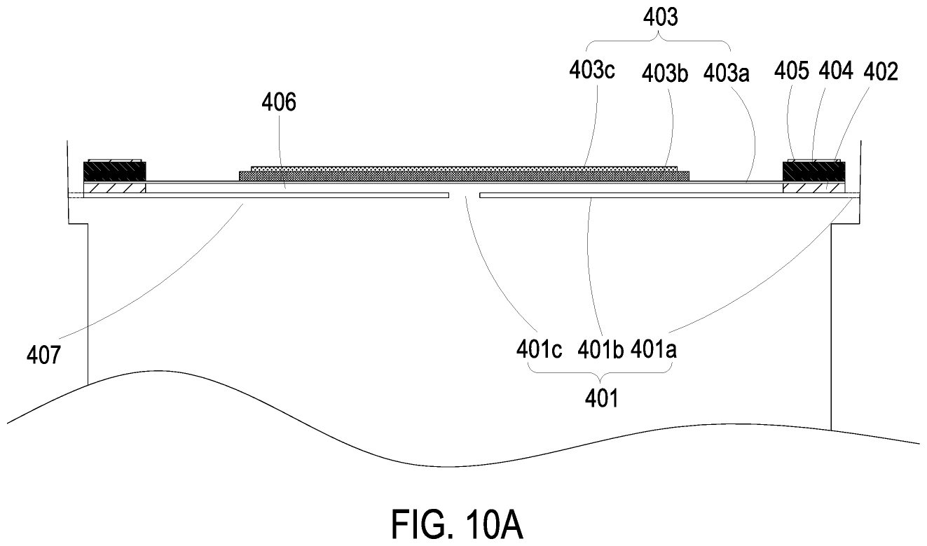

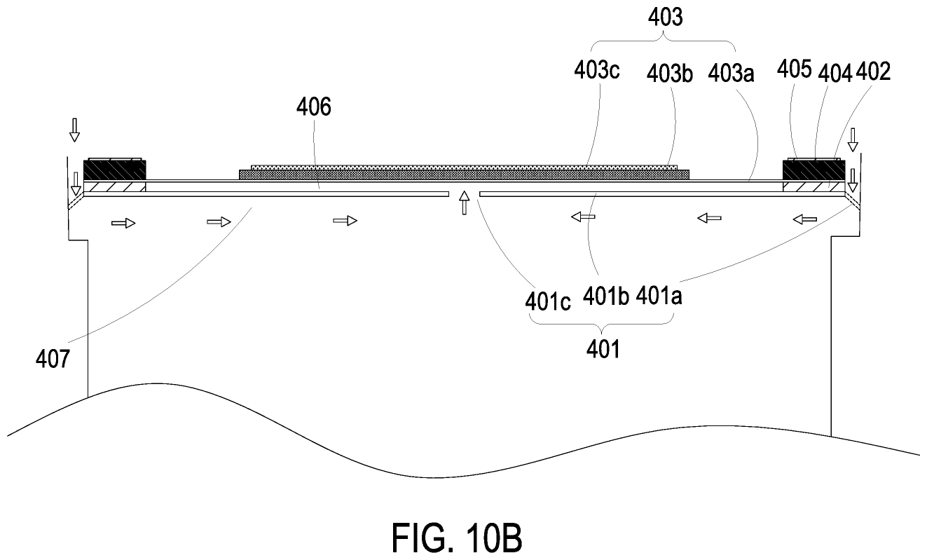

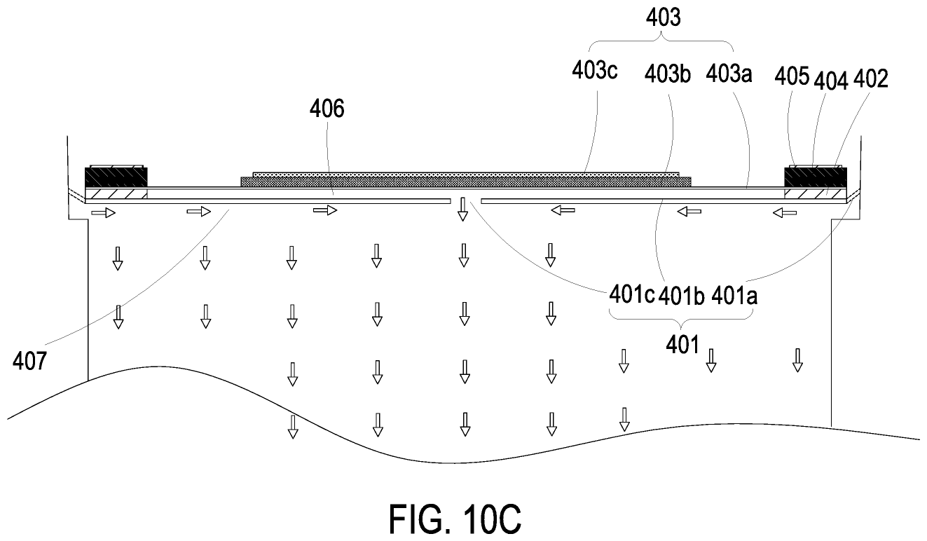

In addition to the miniature pump 30 described above, the gas actuator 224 may be a micro box pump 40 to implement gas transportation. Please refer to FIG. 9 and FIGS. 10A to 10C. The micro box pump 40 includes a nozzle plate 401, a chamber frame 402, an actuating element 403, an insulation frame 404 and a conducting frame 405, which are stacked on each other sequentially. The nozzle plate 401 includes a plurality of connecting element 401a, a suspension board 401b and a central aperture 401c. The suspension board 401b is permitted to bend and vibrate. The plurality of connecting elements 401a is connected to the edge of the suspension board 401b. In this embodiment, there are four connecting elements 401a, which are connected to four corners of the suspension board 401b, respectively, but not limited thereto. The central aperture 401c is formed in the center of the suspension board 401b. The chamber frame 402 is carried and stacked on the suspension board 401b. The actuating element 403 is carried and stacked on the chamber frame 402, and includes a piezoelectric carrying plate 403a, an adjusting resonance plate 403b and a piezoelectric plate 403c. The piezoelectric carrying plate 403a is carried and stacked on the chamber frame 402. The adjusting resonance plate 403b is carried and stacked on the piezoelectric carrying plate 403a. The piezoelectric plate 403c is carried and stacked on the adjusting resonance plate 403b. As the piezoelectric plate 403c is actuated by an applied voltage, the piezoelectric plate 403c deforms to drive the piezoelectric carrying plate 403a and the adjusting resonance plate 403b to bend and vibrate in a reciprocating manner. The insulation frame 404 is carried and stacked on the piezoelectric carrying plate 403a of the actuating element 403. The conducting frame 405 is carried and stacked on the insulation frame 404. A resonance chamber 406 is formed among the actuating element 403, the chamber frame 402 and the suspension board 401b.

FIGS. 10A to 10C schematically illustrate the actions of the micro box pump 40 of present disclosure. First, please refer to FIG. 9 and FIG. 10A. The micro box pump 40 is disposed and fixed via the plurality of connecting elements 401a. An airflow chamber 407 is formed in the bottom of the nozzle plate 401. Then, please refer to FIG. 10B. When the piezoelectric plate 403c of the actuating element 403 is actuated by an applied voltage, the piezoelectric plate 403c is subjected to deformation owing to the piezoelectric elect, and the adjusting resonance plate 403b and the piezoelectric carrying plate 403a are driven to vibrate synchronously. Meanwhile, the nozzle plate 401 is driven to move owing to the Helmholtz resonance effect, and the actuating element 403 moves in a direction away from the nozzle plate 401. Since the actuating element 403 moves in a direction away from the nozzle plate 401, the volume of the airflow chamber 407 at the bottom of the nozzle plate 401 is increased, and a negative pressure is formed in the airflow chamber 407. The air outside the micro box pump 40 is inhaled into the airflow chamber 407 through the vacant spaces among the plurality of connecting elements 401a of the nozzle plate 401 due to the pressure gradient, and is further compressed. Finally, please refer to FIG. 10C. The gas flows into the airflow chamber 407 continuously, and a positive pressure is formed in the airflow chamber 407. Meanwhile, the actuating element 403 is driven to vibrate in a direction toward the nozzle plate 401 in response to the applied voltage, and the volume of the airflow chamber 407 is compressed. The gas in the airflow chamber 407 is pushed and is discharged from the micro box pump 40. Consequently, the gas transportation is implemented.

In an embodiment, the micro box pump 40 is a micro-electromechanical system gas pump produced by micro-electromechanical manufacturing process. The nozzle plate 401, the chamber frame 402, the actuating element 403, the insulation frame 404 and the conducting frame 405 are all produced by a micro-electromechanical surface micromachining technology. Thereby, the volume of the micro box pump 40 is reduced.

According to above description, the present disclosure provides a gas purifying device. The gas detector 2 of the gas purifying device can be detached from the embedding slot 114 of the detector main body 21 configured for use independently. Therefore, the gas detecting module 22 of the gas detector 2 can monitor the air quality around the user anytime and anywhere. Moreover, the gas actuator 224 inhales gas into the interior of the gas detecting module 22 rapidly and stably, so as to increase the monitoring efficiency of the gas sensor 223. Furthermore, since the compartment body 221 is divided into the first gas compartment 221b and the second gas compartment 221c, and the gas sensor 223 and the gas actuator 224 are separated from each other, so that the heat generated by the gas actuator 224 can be blocked, thereby preventing the accuracy of the detection result of the gas sensor 223 from interference. In addition, the gas sensor 223 is prevented from being affected by other components of the device, so that the gas detector 2 may detect air quality anytime and anywhere and have the benefits of rapid and accurate gas monitoring gas.

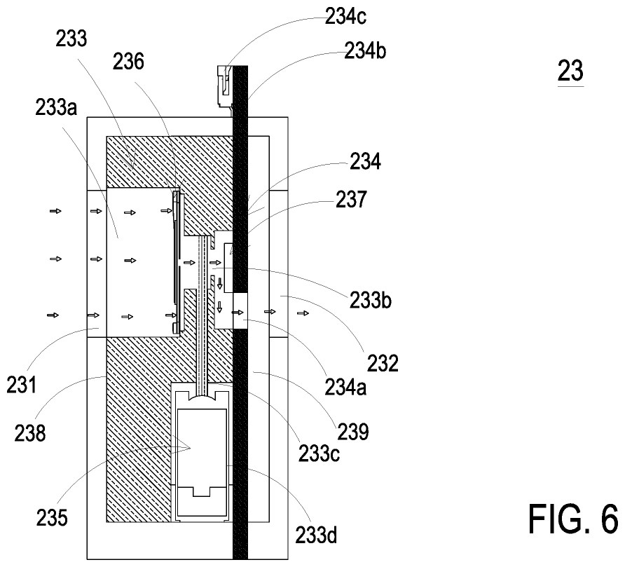

Please refer to FIGS. 3C to 3E and FIGS. 5 to 6. In this embodiment, the gas detector 2 comprises the particulate measuring module 23 for detecting the suspended particles contained in the air. The particulate measuring module 23 is disposed within the chamber 211 of the detector main body 21 and includes an inlet channel 231, an outlet channel 232, a fine particle detecting base 233, a carrying partition 234, a laser transmitter 235, a particulate actuator 236 and a particulate detector 237. The inlet channel 231 is corresponding in position to the second inlet 213 of the detector main body 21. The outlet channel 232 is corresponding in position to the detecting outlet 214 of the detector main body 21. In that, the gas is introduced into the particulate measuring module 23 through the inlet channel 231 and then the gas detected is discharged out through the outlet channel 232. In the embodiment, the fine particle detecting base 233 and the carrying partition 234 are disposed in the particulate measuring module 23. The inner space of the particulate measuring module 23 is divided into a first compartment 238 and a second compartment 239 by the carrying partition 234. The carrying partition 234 has a communication opening 234a for allowing the first compartment 238 and the second compartment 239 to be in fluid communication with each other. The first compartment 238 is in fluid communication with the inlet channel 231, and the second compartment 239 is in fluid communication with the outlet channel 232. Moreover, the fine particle detecting base 233 is adjacent to the carrying partition 234 and disposed within the first compartment 238. In the embodiment, the fine particle detecting base 233 has a receiving slot 233a, a detecting channel 233b, a light-beam channel 233c and an accommodation chamber 233d. The receiving slot 233a is spatially corresponding to the inlet channel 231. The detecting channel 233b is in fluid communication between the receiving slot 233a and the communication opening 234a of the carrying partition 234. The accommodation chamber 233d is disposed in one end of the detecting channel 233b. The light-beam channel 233c is in fluid communication between the accommodation chamber 233d and the detecting channel 233b. The light-beam channel 233c is perpendicular to and intersects the detecting channel 233b. In such way, the inlet channel 231, the receiving slot 233a, the detecting channel 233b, the communication opening 234a and the outlet channel outlet 232 inside the particulate measuring module 23 collaboratively form an airflow path for guiding the gas along a single direction, which is indicated by the arrow in FIG. 6.

In the embodiment, the laser transmitter 235 is accommodated within the accommodation chamber 233d. The particulate actuator 236 is disposed in the receiving slot 233a. The particulate detector 237 is electrically connected to the carrying partition 234 and is disposed on one end of and under the detecting channel 233b. In that, the laser beam of the laser transmitter 235 is transmitted and guided into the detecting channel 233b through light-beam channel 233c, so as to irradiate suspended particles contained in the gas flowing through the detecting channel 233b. When the suspended particles contained in the gas are irradiated to generate scattered light spots, the scattered light spots are projected on a surface of the particulate detector 237 for measuring the sizes and the concentration of the suspended particles contained in the gas. In this embodiment, the particulate detector 237 may be a PM2.5 sensor.

As described in the above, the detecting channel 233b of the particulate measuring module 23 is perpendicular to the inlet channel 231. That is, the position of the inlet channel 231 is disposed directly on the detecting channel 233b to make the airflow path connect to the detecting channel 233b in a straight direction. In that, the airflow resistance on the airflow path is eliminated as much as possible. In the embodiment, the particulate actuator 236 is disposed in the receiving slot 233a to inhale the air from the exterior through the inlet channel 231 without hindrance, so that the gas flows along the straight direction into the detecting channel 233b without hindrance and detected by the particulate detector 237. The efficiency of the particulate detector 237 is enhanced.

Please refer to FIG. 6. The carrying partition 234 further has an exposed part 234b, which penetrates and extends out of the particulate measuring module 23. The exposed part 234b includes a connector 234c disposed thereon to allow a flexible circuit board to be inserted thereinto, so as to provide an electrical connection and signal communication of the carrying partition 234. In one embodiment, the carrying partition 234 may be a circuit board.

The characteristics of the particulate measuring module 23 are described as the above. In an embodiment, the particulate actuator 236 is a miniature pump 30. The structures and operations of the miniature pump 30 are described as the above, and are not redundantly described hereinafter. In other embodiment, the particulate actuator 236 is a micro box pump 40. The structures and operations of the micro box pump 40 are described as the above, and are not redundantly described hereinafter.

Please refer to FIGS. 3E, 6 and 11. The detecting power supply battery 24 is connected to a power source for storing electrical power therein and supplies electrical power to the gas detecting module 22, the particulate measuring module 23 and the detector drive control module 25 as the driving power source. The detecting power supply battery 24 is connected to the power source and may be charged by a wired transmission or a wireless transmission. The detecting power supply battery 24 is electrically connected to the power supply battery 141 of the drive control module 14 through the connection port 115 of the gas purifier 1 (see FIG. 2A) so as to provide electrical power.

Please refer to FIG. 11. The detector drive control module 25 includes a detecting microprocessor 251, an Internet of Things (IoT) communication component 252, a data communication component 253 and a global positioning system component 254. The actuation of the gas detecting module 22 and the particulate measuring module 23 are controlled by the detecting microprocessor 251 and the monitored information is acquired by the detecting microprocessor 251. The detecting microprocessor 251 converts the monitored information into monitored data information and outputs the monitored data information to the Internet of Things communication component 252. The Internet of Things communication component 252 transfers the monitored data information to a network relay station 60 and the network relay station 60 sends the monitored data information to a cloud data processing device 70 by wireless communication transmission for storing and recording. The Internet of Things communication component 252 may be a narrowband Internet of Things device that transmits transmission signals in a narrowband radio communication technology. In some embodiments, the detecting microprocessor 251 transmits the monitored data information to the data communication component 253, and the data communication component 253 transmits the detected data information to an external connecting device 50 for storing, recording or displaying. The data communication component 253 transmits the detected data information through a wired communication transmission or a wireless communication transmission. The wired communication transmission may be at least one selected from the group consisting of a USB, a mini-USB, a micro-USB and combinations thereof. The wireless communication transmission may be at least one selected from the group consisting of a Wi-Fi module, a Bluetooth module, a radio frequency identification module, a near field communication module and combinations thereof. The external connecting device 50 may be at least one selected from the group consisting of mobile phone devices, smart watches, smart bracelets, laptops, tablets and combinations thereof. When the external connecting device 50 receives the detected data information, the detected data information may be transmitted to the network relay station 60, and further transmitted to the cloud data processing device 70 by wireless communication transmission for storing and recording.

From the above descriptions, the present disclosure provides a gas purifying device in combination with a gas detector. The gas purifying device utilizes a gas detection module and a particle detection module to monitor air quality around a user so as to achieve the purpose of carrying by the user and monitoring air quality immediately anytime and everywhere and also achieve the benefits of monitoring rapidly and accurately. Consequently, air quality information is acquired in real time and is provided as a notification to the user in the environment, so that the user can prevent or escape from the injuries and influence on human health caused by the exposure of the harmful substances in the environment. In addition, the gas purifying device utilizes a gas purifying device to achieve the benefits of purifying gas so as to improve air quality.

While the disclosure has been described in terms of what is presently considered to be the most practical and preferred embodiments, it is to be understood that the disclosure needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A gas purifying device, comprising:
a gas purifier comprising a purifier main body, a filter, an air guiding device and a drive control module and configured to purify gas; and
a gas detector comprising:
a gas detecting module comprising a gas sensor and a gas actuator, wherein the gas actuator controls the gas to be guided to the interior of the gas detecting module and the gas is detected by the gas sensor;
a particulate measuring module comprising a particulate detector and a particulate actuator, wherein the particulate actuator controls the gas to be guided to the interior of the particulate measuring module, and the particulate detector measures sizes and concentrations of suspended particles contained in the gas; and
a detector drive control module controlling actuation of the gas detecting module and the particulate measuring module, converting monitored information from the gas detecting module and the particulate measuring module into monitored data information, and outputting the monitored data information.

2. The gas purifying device according to claim 1, wherein the purifier main body has an embedding slot disposed on an exterior thereof, and the gas detector is assembled in the embedding slot to be positioned.

3. The gas purifying device according to claim 2, wherein the gas detector is detached from the embedding slot and configured for use independently.

4. The gas purifying device according to claim 1, wherein the purifier main body has at least one inlet and an outlet disposed on the exterior thereof and has a guiding channel disposed in the interior thereof, the guiding channel is in communication between the at least one inlet and the outlet, the filter is disposed between the at least one inlet and the guiding channel, and the air guiding device is disposed between the outlet and the guiding channel, wherein the air guiding device allows external gas to be inhaled through the at least one inlet, passed through the filter, guided into the guiding channel, and discharged through the outlet.

5. The gas purifying device according to claim 1, wherein the drive control module is disposed in the purifier main body, and a connection port is disposed in the embedding slot and configured for electrical connection to the drive control module, wherein when the gas detector is assembled and positioned in the embedding slot, the gas detector is electrically connected to the drive control module through the connection port so as to be powered thereby.

6. The gas purifying device according to claim 5, wherein the drive control module comprises a power supply battery, a communication component and a microprocessor, wherein the power supply battery is connected to a power source for storing electrical power therein and supplying the electrical power to the microprocessor and the air guiding device, wherein the communication component receives the monitored data information from the detector drive control module and transfers the monitored data information to the microprocessor via a wireless communication technology, the microprocessor converts the monitored data information into a control signal and controls the actuation of the air guiding device to allow the gas purifier to purify the gas, and wherein when the communication component receives a transmission signal from an external connecting device and transfers the transmission signal to the microprocessor via the wireless communication technology, the microprocessor converts the transmission signal into the control signal and controls the actuation of the air guiding device to allow the gas purifier to purify the gas.

7. The gas purifying device according to claim 1, wherein the detector drive control module includes a detecting microprocessor, an Internet of Things communication component, a data communication component and a global positioning system component, wherein the actuation of the gas detecting module and the particulate measuring module are controlled by the detecting microprocessor and the monitored data information is converted and outputted by the detecting microprocessor, the detecting microprocessor outputs the monitored data information to the Internet of Things communication component, the Internet of Things communication component outputs the monitored data information to a network relay station, and the network relay station transfers the monitored data information to a cloud data processing device by a wireless communication transmission for storing and recording.

8. The gas purifying device according to claim 7, wherein the detecting microprocessor outputs the monitored data information to the data communication component, and the data communication component transmits the monitored data information to an external connecting device for storing, recording or displaying, wherein when the external connecting device receives the monitored data information, the external connecting device transfers the monitored data information to the network relay station, and the network relay station transmits the monitored data information to the cloud data processing device by the wireless communication transmission for storing and recording.

9. The gas purifying device according to claim 8, wherein the data communication component transmits the monitored data information to the external connecting device by at least one selected from the group consisting of a USB, a mini-USB, a micro-USB, a Wi-Fi module, a Bluetooth module, a radio frequency identification module, a near field communication module and combinations thereof.

10. The gas purifying device according to claim 6, wherein the external connecting device is at least one selected from the group consisting of a mobile phone device, a smart watch, a smart bracelet, a laptop, a tablet and combinations thereof.

11. The gas purifying device according to claim 1, wherein the gas detector comprises a detecting power supply battery connected to a power source for storing electrical power therein and supplying the electrical power to the gas detecting module, the particulate measuring module and the detector drive control module.

12. The gas purifying device according to claim 1, wherein the detecting power supply battery is connected to the power source for charging and storing electrical power by a wired transmission technology or a wireless transmission technology.

13. The gas purifying device according to claim 12, wherein the gas detector comprises a detector main body and a chamber disposed in the detector main body, wherein the detector main body further comprises a first inlet, a second inlet and a detecting outlet in fluid communication with the chamber.

14. The gas purifying device according to claim 13, wherein the gas detecting module comprises a compartment body and a carrier, the compartment body is disposed under the first inlet of the detector main body, wherein a partition divides the internal of the detector main body into a first gas compartment and a second gas compartment, wherein the partition has a notch for allowing the first gas compartment and the second gas compartment to be in communication with each other, wherein the first gas compartment has an opening, the second gas compartment has a discharging opening, the carrier is disposed under the compartment body, and the gas sensor is packaged on and electrically connected with the carrier, wherein the gas sensor is accommodated in the first gas compartment through the opening, and the gas actuator is disposed in the second gas compartment so that the gas actuator is insulated from the gas sensor, wherein the gas actuator controls the gas to be inhaled via the first inlet, detected by the gas sensor, and discharged via the discharging opening of the compartment body.

15. The gas purifying device according to claim 1, wherein the gas sensor is at least one selected from the group consisting of an oxygen sensor, a carbon monoxide sensor, a carbon dioxide sensor, a volatile organic compound sensor, a bacterial sensor, a virus sensor, a microorganism sensor and combinations thereof.

16. The gas purifying device according to claim 13, wherein the particulate measuring module comprises an inlet channel, an outlet channel, a carrying partition, a fine particle detecting base and a laser transmitter, wherein the inlet channel is corresponding in position to the second inlet of the detector main body, the outlet channel is corresponding in position to the detecting outlet of the detector main body, the particulate measuring module has an inner space divided into a first compartment and a second compartment by the carrying partition, the carrying partition has a communication opening for allowing the first compartment and the second compartment to be in fluid communication with each other, the first compartment is in fluid communication with the inlet channel, and the second compartment is in fluid communication with the outlet channel, wherein the fine particle detecting base is adjacent to the carrying partition and disposed within the first compartment, and comprises a receiving slot, a detecting channel, a light-beam channel and an accommodation chamber, wherein the receiving slot is perpendicularly corresponding to the inlet channel, the particulate actuator is disposed in the receiving slot, the detecting channel is disposed under the receiving slot, and the accommodation chamber is disposed in one end of the detecting channel to accommodate and position the laser transmitter, wherein the light-beam channel is in fluid communication between the accommodation chamber and the detecting channel, and the light-beam channel is perpendicular to and intersects the detecting channel, so as to allow a laser beam of the laser transmitter to irradiate the detecting channel, wherein the particulate detector is disposed on one end of the detecting channel, whereby the particulate actuator allows the gas to flow into the receiving slot through the inlet channel and be transported to the detecting channel, the gas is irradiated by the laser beam of the laser transmitter, the scattered light spots are projected on the surface of the particulate detector for measuring the sizes and the concentrations of the suspended particles contained in the gas, and the gas is discharged out through the outlet channel.

17. The gas purifying device according to claim 16, wherein the carrying partition is a print circuit board, and the particulate detector is electrically connected to the carrier partition, and located at one end of the monitoring channel.

18. The gas purifying device according to claim 1, wherein the particulate detector is a PM2.5 sensor

19. The gas purifying device according to claim 1, wherein the gas actuator and the particulate actuator are micro-electromechanical system gas pumps.

20. The gas purifying device according to claim 1, wherein the gas actuator and the particulate actuator are miniature pumps, respectively, wherein the miniature pump includes:
a gas inlet plate having at least one inlet aperture, at least one convergence channel and a convergence chamber, wherein the at least one inlet aperture allows the gas to flow in, the at least one convergence channel is disposed corresponding to the at least one inlet aperture and guides the gas from the at least one inlet aperture toward the convergence chamber;
a resonance plates assembling on the gas inlet plate and having a central aperture and a movable part, wherein the central aperture is located in the center of the resonance plate and corresponding in position to the convergence chamber, and the movable part surrounds the central aperture, and the region of the periphery of the resonance plate securely attached on the gas inlet plate is the fixed part; and
a piezoelectric actuator aligned with the resonance plate, wherein the piezoelectric actuator comprises:
a suspension plate, wherein the suspension plate is a square suspension plate and permitted to undergo a bending vibration;
an outer frame arranged around the suspension plate;
at least one bracket connected between the suspension plate and the outer frame for elastically supporting the suspension plate; and
a piezoelectric element, wherein a length of a side of the piezoelectric element is less than or equal to a length of a side of the suspension plate, wherein the piezoelectric element is attached on a surface of the suspension plate, wherein when a voltage is applied to the piezoelectric element, the suspension plate is driven to undergo a bending vibration;
wherein a chamber gap is formed between the resonance plate and the piezoelectric actuator, so that the gas from the at least one inlet aperture of the gas inlet plate is converged to the convergence chamber along the at least one convergence channel and flows through the central aperture of the resonance plate when the piezoelectric actuator is driven, whereby the gas is further transferred through a resonance between the piezoelectric actuator and the movable part of the resonance plate.

21. The gas purifying device according to claim 20, wherein the miniature pump comprises an first insulation plate, a conducting plate and a second insulation plate, wherein the gas inlet plate, the resonance plate, the piezoelectric actuator, the first insulation plate, the conducting plate and the second insulation plate are stacked sequentially, wherein the suspension plate has a bulge formed on a surface of the suspension plate opposite to the surface attaching the piezoelectric element, wherein the bulge is formed on the suspension plate by an etching process and is a convex structure integrally formed on the surface of the suspension plate opposite to the surface attaching the piezoelectric element.

22. The gas purifying device according to claim 1, wherein the gas actuator and the particulate actuator are micro box pumps, respectively, wherein the micro box pump comprises:
a nozzle plate having a plurality of connecting elements, a suspension board and a central aperture, wherein the suspension board is permitted to undergo a bending vibration, the plurality of connecting elements are connected to a periphery of the suspension board, and the central aperture is formed in a central position of the suspension board, wherein the nozzle plate is connected by the plurality of connecting elements as being elastically supported, and an airflow chamber is formed at the bottom of the nozzle plate, wherein at least one vacant space is formed among the plurality of connecting elements and the suspension board;
a chamber frame carried and stacked on the suspension plate;
an actuating element carried and stacked on the chamber frame, wherein the actuating element is configured to bend and vibrate in a reciprocating manner in response to an applied voltage, and the actuating element comprises:
a piezoelectric carrying plate carried and stacked on the chamber frame;
an adjusting resonance plate carried and stacked on the piezoelectric carrying plate; and
a piezoelectric plate carried and stacked on the adjusting resonance plate, wherein the piezoelectric plate is configured to drive the piezoelectric carrying plate and the adjusting resonance plate to bend and vibrate in the reciprocating manner in response to the applied voltage;
an insulation frame carried and stacked on the actuating body; and
a conducting frame carried and stacked on the insulation frame;
wherein a resonance chamber is formed among the actuating element, the chamber frame and the suspension plate, wherein when the actuating element is actuated, a resonance of the nozzle plate occurs so that the suspension plate thereof is driven to vibrate and displace in a reciprocating manner, thereby making the gas flow through the at least one vacant space into the airflow chamber and then exhaust through the monitoring channel to achieve transportation of the gas.
